(19) United States
(12) Patent Application Publication
(10) Pub. No.: US 2022/0296121 A1
(43) Pub. Date: Sep. 22, 2022

(54) SYSTEM, METHOD AND ACCESORIES FOR DIELECTRIC-MAPPING

(71) Applicant: Navix International Limited, Road Town, Tortola (VG)

(72) Inventors: Shlomo BEN-HAIM, Milan (IT); Oran GERBAT, Hod-HaSharon (IL); Yehuda LANDAU, Ramat-Gan (IL)

(73) Assignee: Navix International Limited, Road Town, Tortola (VG)

(21) Appl. No.: 17/603,677

(22) PCT Filed: Apr. 16, 2020
(86) PCT No.: PCT/EP2020/060766

§ 371 Date: Oct. 14, 2021

Related U.S. Application Data
(60) Provisional application No. 62/834,463, filed on Apr. 16, 2019.

(51) Int. Cl. A61B 5/0536; A61B 5/0538

(52) U.S. Cl. CPC A61B 5/0536; A61B 5/0538; A61B 5/6852

(57) ABSTRACT

A method of computing a dielectric map is disclosed comprising exciting at least one pair of electrodes according to an excitation scheme, the at least one pair of electrodes comprising at least one pair of in-body electrodes (also referred herein below intra-body electrode) located inside of the examined living body, measuring and recording voltages developing on the in-body electrodes during the excitation according to the excitation scheme, solving an inverse problem to derive a 3D dielectric map from the recorded voltages and optionally providing a 3D image of the body tissues based on the 3D dielectric map. Methods are also disclosed that combine intrabody electrodes and surface electrodes secured to the body or use only surface electrodes. Embodiments encompass the use of constraints in deriving the 3D dielectric map and combining measurements made at different locations inside the body with moving intrabody electrodes. Disclosed methods are not limited to methods including exciting and measuring on the body but also extend to methods of processing data previously obtained to derive the 3D map.

### FIELD AND BACKGROUND OF THE DISCLOSURE

The present disclosure relates to mapping physical properties of a body part or organ, for example in medical imaging and, more specifically, but not exclusively, to systems and methods for dielectric mapping and imaging, e.g., for the construction of body tissues and organs.

Electrical Impedance Tomography (EIT) systems and methods of medical imaging, as is known in the art, are implemented by deploying electrodes at the body's surface of a subject, injecting electrical excitation to some of the employed electrodes, measuring the electrical signals received at other employed electrodes, calculating, based on the measured signals, 3D image(s) of tissues and organs inside the body and providing a display of the calculated 3D images. EIT techniques are based on the fact that muscle and blood conduct the applied currents better than fat, bone, or lung tissue and are therefore able to resolve different tissue types. However, current approaches suffer from low resolution of the obtained images.

There is a need for system and method that provide imaging of body organs and lumens with improved accuracy.

### SUMMARY

In overview, the disclosure provides a method of generating a dielectric map of a region of an organ of a human or animal body using intrabody electrodes that were or are disposed inside or adjacent the region. In some embodiments, the intrabody electrodes are moved through the region and dielectric maps mapping different parts of the region, each part being mapped using the electrodes in a different position or orientation, are combined, for example, stitched together, to generate the dielectric map of the region. The dielectric map of the or each region provides a spatial distribution of one or more dielectric properties of tissue in the mapped region. The tissue may be, for example, blood, muscle, bone, nerve, and/or fat tissue. Examples of dielectric properties that may be mapped in the dielectric map includes conductivity, complex conductivity, real or imaginary part of conductivity, permittivity, complex permittivity, real or imaginary part of permittivity, impedance, etc.

In a first aspect, a method of generating a dielectric map of one or more dielectric properties in a region of an organ of a human or animal body is disclosed. The method comprises accessing a first plurality of data sets, each data set of the first plurality comprising measured voltage data indicative of voltages measured at a respective second set of one or more electrodes in response to electric fields in the region generated by currents applied to a respective first set of one or more electrodes. The first plurality of data sets are or were thus obtained using respective pairs of sets of electrodes, one for generating field(s) in response to applied currents and the other for measuring voltages due to the generated fields. The first and second sets of electrodes comprise electrodes disposed on a tool located at a first location in the region at the time of measurement. The first and second sets of electrodes may have electrodes in common. Position data indicative of positions of the electrodes in the respective first and second sets of electrodes relative to the tool are also accessed. In this and in any other aspect of the disclosure, accessing position data and accessing a plurality of data sets can make part of a single step, or carried out in different steps. The method further comprises generating at least a portion of the dielectric map by computing a first spatial distribution of one or more dielectric properties in the region using the first plurality of data sets and the position data.

In some embodiments, the method may further comprise:
determining the position of the tool in a reference frame and
positioning the dielectric map in the reference frame based on the determined position.

The reference frame may be fixed relative to the body, or fixed relative to another tool. Alternatively in some embodiments the reference frame may not be fixed relative to any known landmark or the body, and the position of the tool may be determined using a voltage to position mapping technique as described below.

Determining the position of the tool in a reference frame may comprise generating a global dielectric map of a portion of the body comprising the region, for example as described in the first aspect but with electrodes disposed in fixed relation to the body, for example, fixed to the skin of the patient or to a belt or garment worn by the patient, and determining the position of the tool based on the global dielectric.

In all of the disclosed aspects and embodiments, measured voltage data may be measured voltages but also other quantities indicative of voltage, such as electric field measurements, impedance measurement and any other measurement indicative of a voltage developed at the second set of electrodes. The currents are typically time varying currents, for example varying at a given frequency or within a frequency range, for example to generate radio frequency (RF) fields, more specifically within a frequency range of 1 to 1000 kHz, preferably 1 to 400 kHz or 1 to 100 Hz. Frequencies up to 4 MHz may also be used. It will be understood that the currents may be fixed in amplitude and/or frequency, either to be the same for all field generating electrodes, or specifically assigned in advanced to certain electrodes, so that the currents are known in advance. In other cases, respective current values may be received with the data set, based on knowledge of the currents applied or measured. The position data may be explicit in terms of positions, for example coordinates, of the electrodes. Alternatively or additionally, the position data may be implicit, for example, in terms of an identifier of an electrode having a position (e.g., in respect to a frame of reference fixed to the tool). In another example the identifier may be implicit, for example, the place of the electrode in a known sequence of electrodes of known positions relative to the tool.

In some examples the positions of the electrodes may be defined in a coordinate system that is not fixed to any known reference frame, such as a reference frame external to the body, fixed to the body or fixed to a tool. The electrode positions may instead be defined in a coordinate system that is independent of a tool or body and is not defined relative to an external reference outside of the body. A common reference frame may be determined using electrodes that move to different positions and take voltage measurements at different times. A coordinate system is determined in which the positions of all the electrodes at all the different times can be found, thereby providing a common reference frame for all the electrode positions that does not rely on landmarks inside or outside of the body to define the coordinate system, or on a reference frame fixed to the body or to the tool. One particular example of finding a common reference frame for moving electrodes is using the “V-to-R” or “measurement-to-location” navigation and imaging system as described in WO2019034944A1, in which voltage measurements made using electrodes on a tool are used to determine a position of those electrodes in a common reference frame. This is done by transforming a cloud of voltage measurements (referred to as the V-cloud) that are acquired at different sets of positions of the electrodes, into positions of the electrodes at which the measurements were taken (referred to as the R-cloud). In some examples, one way of finding the common reference frame involves making a plurality of voltage measurements for a plurality of different respective locations of the electrodes, such that enough points exist in the V-cloud (there are enough measurements at different electrode positons) to produce a voltage-to-position mapping or transformation of sufficient accuracy. In other words, the electrodes may be repeatedly moved to different positions and voltage measurements made for the electrodes at those positions until enough measurements have been made to generate an R-cloud (by transforming the voltage measurements (the V-cloud)) with a sufficiently large number of points. The transformation to the R-cloud may then be used to find the position of each electrode in a common reference frame for the existing voltage measurements and for future measurements. A reference frame may be defined based on the cloud of positions, for example with an origin at the centre of the R-cloud, and so the positions of the electrodes for each voltage measurement can be determined in this reference frame. Whilst this frame of reference may not be known, for example relative to an external reference or relative to any other fixed reference, the common frame of reference is the same for all voltage measurements taken at all the different positions of the respective electrodes. The positions of the electrodes when subsequent voltage measurements made (e.g. when a tool carrying the electrodes is moved to a new position) can then be determined in the common reference frame using the transformation.

Electrodes may be used to generate respective independent fields by exciting the respective fields (using the respective first sets of electrodes) in sequence and/or the respective independent fields may be generated by exciting some or all of the electrodes simultaneously but at different respective frequencies. In the latter case, the measurement at the corresponding second set of electrodes would be combined with signal processing to take measurements at the relevant frequency. For example, in some embodiments, a plurality of electrodes, possibly all but one of the available electrodes, each excite a field with a respective frequency and measurement of all these fields is done at the same sensing electrode (that constitutes the second set of electrodes) for all data sets. In this example, there is thus a data set for each of the plurality of electrodes, each having one of the plurality of electrodes constituting the first set of electrodes and the single sensing electrode constituting the second set of electrodes, with the electrodes disposed, for example as described below. Generally, in different data sets, the electrodes may be assigned to the first and second sets in different ways. Each data set thus represents an independent measurement and may include data acquired at different points in time or at different frequencies.

In some embodiments, the first and second sets of electrodes each consists of electrodes disposed on the tool, that is, all of the electrodes used for field generation and measuring are disposed on the tool. In other embodiments, some of the electrodes may be disposed in a fixed relationship with the body on a different tool disposed inside the body or on the outside of the body, e.g., attached to the skin or worn on a belt or garment (surface electrodes). The electrodes being disposed in a fixed relationship with the body mean that the electrodes may move as the body moves, for example due to breathing. The electrodes may be arranged on the tool in a number of ways, for example in line, in some case along a longitudinal direction of the tool. In other cases, the electrodes may have a three-dimensional arrangement on the tool. The tool may be a catheter, for example a basked catheter, scalpel, guide wire, suture or any suitable surgical instrument. The tool may carry as many as 25 or more electrodes, in particular in case of a basket catheter, or as little as four or even two electrodes. For example, the tool may carry 12 electrodes. Where applicable, any number of static (surface or intrabody) electrodes may be used in combination with the electrodes on the tool.

In some embodiments, one or more ground electrodes are also provided in conjunction with the first and second sets of electrodes, and a voltage measurement taken using each electrode of the second set of electrodes is a voltage difference between a voltage measured at that electrode and a voltage measured at the ground electrode. Whilst the first set of electrodes functions as a field source, i.e. supplying an electric field, the ground electrode functions as a field sink. A single ground electrode may be used in conjunction with the first set of electrodes, or a different respective ground electrode may be used for each respective different frequency of the first set of electrodes (when different ones of the first set of electrodes are excited at different respective frequencies). The ground electrode(s) may be a surface electrode positioned on the surface of the body, such as attached to the skin of a patient, or the ground electrode(s) may be disposed on the tool along with the first and second sets of electrodes. The second of these options, i.e. the ground electrode being disposed on the tool along with the first and second sets of electrodes, is particularly advantageous. This is because voltages between each of the second set of electrodes and the ground electrodes are local (since the electrodes are close together and possibly in a fixed relationship to one another, depending on the tool) and so the measurements are less affected by long range noise (such as movement of the body due to breathing).

In some embodiments, the method comprises generating two dielectric maps as described above, wherein each map is generated from accessed data comprising measurements made when the tool is or was in the region at a different location at the time of measurement. In some such embodiments, the method further comprises accessing an indication of a displacement between said different locations and combining the two maps using the indication of the displacement.

In some embodiments, the method comprises generating two spatial distributions of one or more dielectric properties in a region of an organ as described above, wherein each spatial distribution is generated from accessed data comprising measurements made when the tool is or was in the region at a different location at the time of measurement. In some such embodiments, the method further comprises accessing an indication of a displacement between said different locations, combining the two spatial distributions using the indication of the displacement, and generating a map based on the combined spatial distribution.

For example, in some embodiments, the method comprises accessing a second plurality of data sets, each data set of the second plurality comprising measured voltage data indicative of voltages measured at a respective fourth set of electrodes in response to the electric fields generated by currents applied to a respective third set of electrodes to generate electric fields in the region. The method also comprises, either as part of the accessing the plurality of data sets or as a separate step, accessing position data indicative of positions of the electrodes in the respective third and fourth set of electrodes relative to the tool, wherein the respective third and fourth sets of electrodes comprise electrodes disposed on the tool and the tool is or was in the region at a second location at the time of measurement. The method in these embodiments further comprises accessing an indication of a displacement of the tool between the first and second locations of the tool, computing a second spatial distribution of one or more dielectric properties in the region using the second plurality of data sets and the position data indicative of positions of the electrodes in the respective third and fourth set of electrodes and combining the first and second spatial distributions using the indication of the displacement to generate at least a portion of the dielectric map.

In some embodiments, the method comprises computing the second spatial distribution using the first spatial distribution, a correspondence between locations in the first spatial distribution and locations in the second spatial distribution and the second plurality of data sets. For example, computing the second spatial distribution may comprise setting values of the one or more dielectric properties at respective locations in the second spatial distribution to values of the one or more dielectric properties at corresponding respective locations in the first spatial distribution as a starting spatial distribution and iteratively adjusting the second spatial distribution using an error signal.

Combining the first and second spatial distributions may comprise combining, for example averaging, values of the one or more dielectric properties at respective locations in the second spatial distribution with values of the one or more dielectric properties at corresponding respective locations in the first spatial distribution. The method may comprise determining the correspondence between locations in the first spatial distribution and locations in the second spatial distribution using the indication of the displacement.

Accessing the indication of displacement may comprise computing a value indicative of the displacement, and then accessing the computed value. Computing the value indicative of the displacement may comprise computing a cross-correlation between the first and second spatial distributions and determining the indication of displacement between the first and second spatial distributions as the displacement at which the cross-correlation exceeds a comparison value, preferably the displacement for which the cross-correlation has a maximum value. Alternatively or additionally, computing the value indicative of the displacement may comprise accessing first voltage values measured at the electrodes on the tool at the first location in response to at least three respective mutually non-parallel electric fields that have been generated in the region from outside the body, accessing second voltage values measured at the electrodes on the tool at the second location in response to the at least three respective mutually non-parallel electric fields, computing an electric field gradient for each mutually non-parallel electric field using the first voltage and computing the indication of the displacement using the first and second voltage values and the computed electric field gradients.

Alternatively or additionally, the method may comprise computing the value indicative of the displacement using data collected from electrodes placed in a fixed relationship to the body. For example, the data collected from static electrodes may comprise voltages recorded at the static electrodes in response to currents applied to static electrodes. For example, the static electrodes may be disposed on the body and/or on a tool that has been placed in a stationary position inside the body, preferably inside the organ.

In some embodiments, the method may comprise determining the respective positions of the tool at the first and second locations in a reference frame fixed relative to the body and determining the indication of the displacement using the determined positions.

For example, determining the respective positions of the tool at the first and second locations may comprise generating a third and fourth (global) spatial distributions of one or more dielectric properties in a body part comprising the region when the tool is, respectively, in the first and second positions. Each of the global spatial distributions may be generated by a method as described above, but with the electrodes placed in a fixed relationship to the body, for example, fixed to the skin of the patient or worn on a belt or garment, or disposed on a tool that has been placed in a stationary position inside the body, preferably inside the organ.

Specifically, determining the respective positions may comprises analyzing each of the third and fourth spatial distributions to detect one or more electrodes on the tool in each of the third and fourth spatial distribution and determining the respective positions using the positions of the one or more electrodes in the respective spatial distribution. Alternatively or additionally, determining the respective positions may comprise determining the tool positions at the first and second locations using cross-correlations. Specifically this may comprise:
computing a cross-correlation between the first and third spatial distributions;
determining the position of the tool at the first location using the displacement between the first and third spatial distributions at which the cross-correlation exceeds a comparison value, preferably the displacement for which the cross-correlation has a maximum value;
computing a cross-correlation between the second and fourth spatial distributions; and
determining the position of the tool at the second location using the displacement between the second and fourth spatial distributions at which the cross-correlation exceeds a comparison value, preferably the displacement for which the cross-correlation has a maximum value.
   In some embodiments, determining the respective positions may comprise:
accessing first voltage values measured at the electrodes on the tool at the first location in response to at least three respective mutually non-parallel electric fields that have been generated in the region by a seventh set of electrodes that have been disposed in a fixed relationship with the body;
accessing second voltage values measured at the electrodes on the tool at the second location in response to the at least three respective mutually non-parallel electric fields;
accessing a voltage to position mapping with the first voltage values to determine a first one of the respective positions;
accessing the voltage to position mapping with the second voltage values to determine a second one of the respective positions. The electrodes of the seventh set of electrodes may have been disposed on the body and/or are disposed on a tool that has been placed in a stationary position inside the body, preferably inside the organ.

In the various described aspects and embodiments, the first and second (and other) spatial distributions may be defined on a respective non-uniform mesh and combining the first and second spatial distributions comprises transforming each of the first and second spatial distribution to be defined on a common mesh, having corresponding points in the combined region of the first and second spatial distributions. For example, the common mesh may be uniform, preferably Cartesian, in the combined region. In embodiments where the first distribution is used as a starting point for the second distribution, when the first and second spatial distributions are defined on a respective non-uniform mesh, computing the second spatial distribution may comprise transforming the first spatial distribution to be defined on the mesh of the second spatial distributions. For example, the first distribution may be transformed to a regular or uniform, for example cartesian mesh, and may then be used to initialize the overlapping region of the second distribution, transforming from the regular or uniform mesh to the mesh of the second distribution after suitable translation to account for the displacement between the first and second locations.

In some embodiments, computing one or more of the spatial distributions comprises accessing constraint data characteristic of a spatial distribution of one or more dielectric properties of the tool disposed in the electric fields and using the constraint data to compute the one or more of the spatial distributions. Specifically, the constraint data may comprise one or more of: a configuration of two or more of the electrodes disposed on the tool; a shape of one or more of the electrodes disposed on the tool; a distance between two electrodes disposed on the tool; and respective distances between pairs of electrodes disposed on the tool. The tool may comprise one or more conductive elements and the constraint data comprises one or more of: a configuration of two or more of the conductive elements; a shape of one or more of the conductive elements; a distance between two conductive elements; and respective distances between pairs of electrodes disposed on the tool. The one or more conductive elements may comprise the electrodes and one or more other conductive elements. Alternatively or additionally the constraint data may comprise a distribution of dielectric properties of a dielectric (non-conducting) portion of the tool.

The disclosure further extends to a method of generating an image, the method comprising generating a dielectric map as described above and assigning a tissue type, colour or greyscale value to locations in the dielectric map based on the value of the one or more dielectric properties at the one or more location. The method may comprise converting the map to a coordinate system suitable for display.

Also disclosed is a system for generating a dielectric map, the system comprising a processor configured to implement a method as described above and a memory for storing the plurality of data sets and the dielectric map or maps. Where applicable, the system may also comprise a display for displaying the medical image. In some cases, the system may comprise an interface for connecting the system to the electrodes.

The methods described above are specifically independent of how and when the data was acquired, In some cases, methods as described above may comprise placing a tool in the region, defining a plurality of pairs of sets of electrodes, generating an electric field in the region using a first set of each pair and measuring a voltage at a respective second set of each pair to generate a plurality of data sets; and accessing the plurality of data sets, each data set comprising current data indicative of currents applied to the first set of electrodes of a respective pair of sets and voltage data indicative of voltages measured at the second set of electrodes of the respective pair of sets. For example, accessing a plurality of data sets may comprise defining a plurality of pairs of sets of electrodes, generating an electric field in the region using a first set of each pair; measuring a voltage at a respective second set of each pair to generate a plurality of data sets.

Also disclosed is a method of generating a dielectric map of one or more dielectric properties in a region of an organ of a human or animal body, the method comprising: accessing a plurality of data sets, each data set comprising voltage data indicative of voltages measured at a respective second set of electrodes in response to electric fields generated in the region by currents applied to a respective first set of electrodes; accessing constraint data characteristic of a spatial distribution of one or more dielectric properties [conductivity, complex conductivity, permittivity, complex permittivity] of a tool disposed in the electric fields; and computing the dielectric map as a spatial distribution of one or more dielectric properties in the region using the plurality of data sets and the constraint data.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE DRAWINGS

Specific embodiments of the present disclosure are described below by way of example and with reference to the accompanying drawings in which:

FIG. 1 schematically depicts deployment of a set of electrodes on and in a body;

FIG. 2A is a schematic illustration of a catheter useful in the present disclosure;

FIG. 2B is a diagrammatic presentation of a basket catheter for dielectric mapping;

FIG. 3 schematically depicts an electric field generator/measurer;

FIG. 4 is a schematic block diagram of a system for dielectric mapping and imaging;

FIG. 5 is a flow chart of a process for converting a collection of measured voltages on a set of electrodes into a 3D map and image;

FIG. 6 is a flow chart of a process for dielectric mapping and imaging;

FIG. 7 is a flow chart of a process for iteratively solving the inverse problem;

FIG. 8 is a flow chart of a process of combine maps corresponding to different catheter positions;

FIG. 8A illustrates the stitching together of a plurality of maps, including combining maps in overlapping areas;

FIG. 9 is a flow chart of a process of computing a map corresponding to one catheter position based on another map corresponding to another catheter position and the overlap between the maps;

FIG. 10 is a flow chart of a process of computing a map displacement and combining maps using the displacement;

FIG. 11 is a flow chart of a process of applying a displacement to map defined on a non-uniform mesh;

FIG. 12 is a flow chart of a process of calculating a displacement using externally applied field gradients; and

FIG. 13 is a flow chart of a process of calculating a displacement using a dielectric map obtained using static electrodes, for example surface electrodes.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

The present disclosure relates to conductivity mapping, for example for dielectric mapping or imaging, e.g., for reconstruction of body tissues and organs. For the sake of simplicity, conductivity or conductance is described below as an example of a mapped quantity in a dielectric map, but it will be appreciated that any other dielectric property, for example as set out above, may be mapped instead and any such quantity can be used in place of conductivity where conductivity is recited in the description that follows. A dielectric map will be understood to represent a spatial distribution of a dielectric property of the mapped region.

In the following description, numerous specific details are set forth in order to provide a thorough understanding of the disclosure. However, it will be understood by those skilled in the art that the present disclosure may be practiced without these specific details. In other instances, well-known methods, procedures, and components have not been described in detail so as not to obscure the present disclosure. The terms ‘injecting signal’, ‘injecting current’, ‘exciting signal’ and ‘exciting current’ will be all used herein after to describe signals provided to electrodes used in the process of imaging as described below.

It will be understood that the present disclosure may be embodied in a system, a method, and/or a computer program product. The computer program product may include a computer readable storage medium (or media) having computer readable program instructions thereon for causing a processor to carry out aspects of the present disclosure.

The flowchart and block diagrams in the Figures illustrate the architecture, functionality, and operation of possible implementations of systems, methods, and computer program products according to the disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In the following detailed description, the terms catheter may refer to any physical carrier of one or more electrodes for insertion of the one or more electrodes into a living body—for example: endoscope, colonoscope, enteral feeding tube, stent, graft, etc. More generally, a tool for insertion into a body may be read in place of “catheter” in what follows. The electrodes on such a catheter or tool may be referred to as intra-body electrodes or in-body electrodes. A catheter or tool may include or may be, for example: a guidewire with electrodes, a micro catheter with electrodes, a sheath with electrodes, a suture thread with electrodes, a spiral catheter with electrodes, a basket catheter with electrodes or a pig tail catheter with electrodes.

The following detailed description is made with reference to voltage measurements. However, it should be noted that embodiments of the present disclosure are not limited to voltage measurements and may deploy other measurements, such as current and/or impedance measurements. Impedance measurements may be obtained from voltage and current measurements on the one or more electrodes. Voltage and current measurements may be real-valued or fully complex.

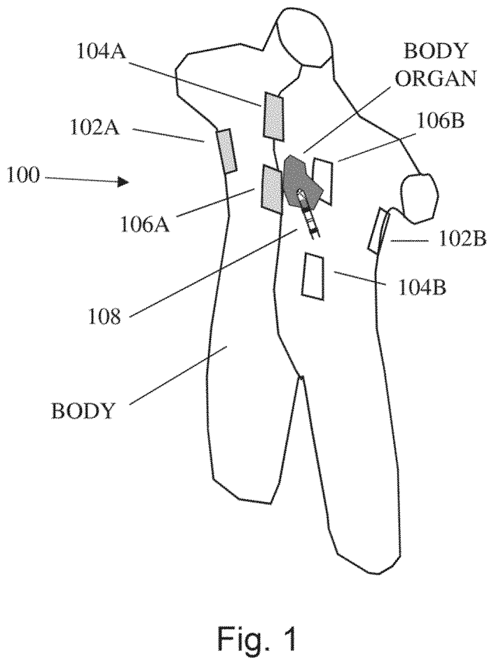

Reference is now made to FIG. 1, which schematically depicts deployment of a set of electrodes 100 on and in a body. In this example, three pairs of surface electrodes (or surface pads) are shown: 102A/102B, 104A/104B and 106A/106B. The pairs of surface electrodes may be disposed on the body substantially at antipode locations. In some embodiments, a smaller or larger number of surface electrodes may be used, and their number may be even or odd. Additionally, set of electrodes 100 comprises intra-body electrodes 103. In the depicted embodiment, the intra-body electrodes are disposed on catheter 108. Catheter 108 may be insertable into a patient's body. In some embodiments, the intra-body electrodes may be carried by more than one catheter, for examples, two electrode-carrying catheters may be inserted into the patient's body, and used for generating an image as described below. In some embodiments, surface electrodes may be replaced with stationary intra-body electrodes or omitted altogether. In some embodiments particularly useful for imaging the heart, specifically the left or right atrium, stationary intra-body electrodes may be placed in the coronary sinus, for example on a catheter that is stationary during imaging.

Surface electrodes 102A/102B, 104A/104B and 106A/106B may be connected to signal source(s) that is/are adapted to inject (or excite) electrical signals in desired strength, frequency and phase.

Voltages developing on the surface electrodes and/or the intra-body electrodes during the excitation may be measured when the intra-body electrodes are actively moved (e.g., by a physician during a medical procedure) around a region of interest (or inside it or along it, etc.)—e.g., around or inside a tissue to be imaged. In some cases, there may be several regions of interest, and the intra-body electrodes may be “dragged” from one to another, back and forth. For example, inside a left atrium there are many structural features that may be of interest, e.g., the openings of the pulmonary veins (which are of high interest for treating atrial fibrillation), the left atrial appendage, the mitral valve, etc. The catheter may be guided to visit all of them (and especially those relevant to an ongoing treatment), and so the image quality at these regions and their vicinity may be improved, as described below. Where reference is made herein to a vicinity, and specifically a vicinity of a region, it would be understood that this refers to a volume of space near to or surrounding the region. As such, a tool placed in the vicinity of a region may be placed near to the region, and may be for example less than 5 cm away from the region, optionally less than 2.5 cm away from the region, preferably less than 1 cm away from the region.

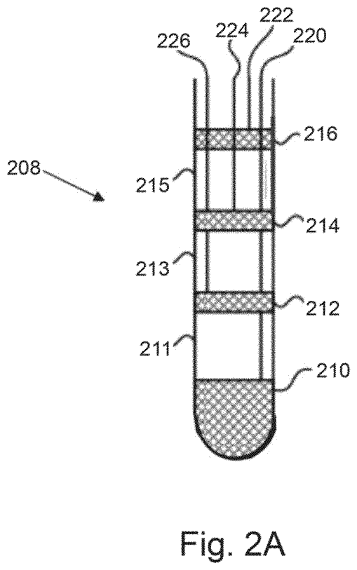

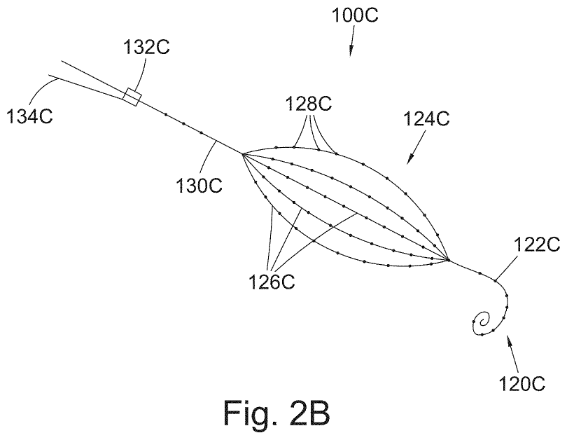

Reference is now made also to FIG. 2A, which is a schematic illustration of catheter 208. Catheter 208 may be, in some embodiments, identical or substantially identical to catheter 108 of FIG. 1. Catheter 208 may comprise one or more electrodes (also referred to herein as intra-body electrodes or in-body electrodes), and in the drawn example four electrodes 210, 212, 214, 216. Each of the electrodes may have connection wire 220, 226, 224, 222, respectively, to enable connecting to electrical excitation unit, such as electric field generator/measurer, e.g. as described with respect to FIG. 3 hereinafter. Electrodes 210, 212, 214, 216 may be disposed spaced from each other along the longitudinal axis of catheter 208 by longitudinal distances 211, 213, 215. The longitudinal distances may be, for example, in the range of lower than 1 millimeter or few millimeters and up to 1-2 cm or up to 4-6 cm between the farthest intra-body electrodes. In some embodiments, the electrodes may be arranged in pairs spaced about 2-3 mm apart, with about 8 mm between pairs. The electrodes may have a length of 1-2.5 mm. In some embodiments, the electrodes may be annular in shape and may be disposed across the catheter with their outer surface substantially flush with the catheter. In some embodiments, these annular electrodes may be dimensioned and spaced as described above. In some embodiments it may be beneficial to have the electrodes spaced apart by a distance that is in the magnitude of order of the size of the scanned organ, or less.

FIG. 2B is a diagrammatic presentation of a basket catheter 100C. Basket catheter 100C may have a pigtail catheter portion 120C, with a plurality of electrodes 122C, optionally arranged in pairs, e.g., 3 or 4 electrode pairs. Basket catheter 100C further includes a basket portion 124C. The basket portion may comprise a plurality of strands 126C, for example, 8 strands or more, usually 12 strands or less, e.g., between 8 to 12 strands. Each strand 126C may include a plurality of electrodes 128C, optionally arranged in pairs.

Basket catheter 100C may further include a proximal catheter portion 130C. In some embodiments, proximal catheter portion is blind, i.e., with no electrodes. In some embodiments, proximal catheter portion 130C may include one or more electrodes, for example, 3 electrodes.

Basket catheter electrode 100C may, in some arrangements, include a chip 132C. In other arrangements, the electrodes are electrically connected to apparatus outside the body by conductive leads. The chip may receive conductive wires (not shown here) connecting the chip to each electrode of the basket catheter electrode 100C, including the electrodes at the proximal catheter portion, and the catheter portion (128C) and at the pigtail catheter portion (122C).

Chip 132C may include a D2A device, transforming digital data to analog signals. The D2A may be used to receive digital data through communication line 134C, and transferring them to analog signals, and transmit the analog signals to the electrodes. In some embodiments, the digital data includes a different set of instructions for each of the electrodes (or for different electrode groups), multiplexed so that each channel carries data with instructions to one of the electrodes. The chip may also include a demux, for demultiplexing the multiplexed signals received, and sending each set of instructions only to the electrode to which the instructions are addressed.

Chip 132C may include an A2D device, transforming analog signals to digital data. For example, to receive measurement results from the electrodes, and digitizing them, to send digitized measurement results through the communication line, for example, to a controller configured to receive the measurement results and analyze them (e.g., control unit 402 and/or controller 404). In some embodiments, some or all of the analysis is done at the chip, and the analysis results are sent via the communication line. Chip 132C may also include a multiplexer, for multiplexing digitized measurement results for sending via a single communication line 134C.

It should be noted that although chip 132C is disclosed in connection to FIG. 2B, it may be included in any catheter or medical device described herein or otherwise.

Schemes of electrical excitations of surface electrodes and/or intra-body electrodes (also referred herein as excitation scheme or scheme of excitation) yield voltages measurable on one or more of the electrodes. The voltage readings (voltages measured on one or more surface electrodes and/or intra-body electrodes) may be used to reconstruct a spatial distribution of the electrical conductivity of tissues through which the electrical signals pass (may be referred to herein as 3D conductivity map). Schemes of excitation may comprise one or more of: selection of the transmitting electrode(s), selection of the frequency of the transmitted signals, selection of the amplitude of each of the transmitted signals, selected duration of the transmission, selection phase differences (or de-phasing) between signals transmitted concurrently from two or more electrodes at a same frequency, and the like. It will be noted that excitation schemes may comprise sets of signal frequencies (transmission frequencies) that may be selected to support one or more needs such as operating in different frequencies to cover different transmissivities of the body tissues along a certain signal path, thereby collecting more information of the tissue's shape. In another example, transmission frequencies may be selected to enable good separation between the transmitted and the received signal, or good separation between signals transmitted concurrently from different electrodes. While separating between signals transmitted concurrently from different electrodes may be achieved with signals separated from each other even in a few kHz, covering different transmissivities may benefit from large frequency differences, for example, frequencies spanning the frequency range between 10 kHz and 100 KHz.

Transmitted signals may be transmitted from one or more of the electrodes, and voltages developing on one or more of the electrodes during the excitation may be received and recorded for further processing. Preferably, voltages developing on all the electrodes are recorded. The voltages may be indicative of the conductivity of body tissues through which the signal passed. Since the conductivity along any electrical path of a signal is indicative of the nature of the tissue along that path, the more different signal paths are sampled, the richer is the data on the nature of the tissues, and a more accurate image (e.g., of higher resolution) may be produced from that data. Accordingly, excitation schemes may be used to invoke transmission from, for example, at least one of the intra-body electrodes and the resulting voltages developing on at least all of the surface electrodes may be recorded, thereby providing, in the example of FIG. 1, indication of six different conductivities, which are indicative of the conductivity of the body tissues along six respective signal paths. The paths along which transmitted signals pass are not known, as the signals do not travel in straight lines, but mainly along paths of minimal resistivity. Yet, the large number of measurements of spatial conductivity values, which may represent, for a large number of points in the examined body organ, measurements of more than one signal path that passes through a certain point, enables reconstructing a detailed 3D map of conductivity values, which may be translated to a 3D image of the imaged tissue (e.g., of the organ).

In some embodiments, excitation schemes may be used to invoke transmission from at least one of the intra-body electrodes and the resulting voltages developing on the remaining intra-body electrodes may be recorded, thereby providing, in the example of FIG. 1, indication of four different signal paths, which are indicative of the conductivity of the body tissues along the respective paths.

Additionally, one or more transmitted signals may be transmitted from at least one of the surface electrodes and the resulting voltages developing on the other surface electrodes may be measured and recorded, thereby providing conductivity information related to signal paths through body tissues extending between the transmitting surface electrode and the at least one receiving surface electrode, which may provide indication of the tissues of the body closer to the body surface. In some arrangements, signals may be transmitted from (i.e. current injected at) one or more of the surface electrodes and measured at one or more of the intra-body electrodes.

In some embodiments, at least some of the excitations may be by electrode pairs, transmitting simultaneously at the same frequency and in opposite phases. In some embodiments, such electrode pair may consist of two surface electrodes or two intra-body electrode electrodes. In some embodiments, such an electrode pair may consist of one intra-body electrode and one surface electrode.

In some embodiments, at least some of the excitations may be by electrode groups of three or more electrodes, transmitting simultaneously at the same frequency and in controlled phase relations between them. In some embodiments, each such electrode group may consist of intra-body electrodes or surface electrodes. In some embodiments, one or more of the groups may include both an intra-body electrode and a surface electrode.

As mentioned above, processing of the measured voltages on the various electrodes may be used, additionally to the creation of a database (or plurality of data sets) of 3D measurements (from which a 3D conductivity map may be produced, as is explained below), also for tracking and positioning the catheter inside the body. Tracking and positioning of the catheter inside the body may be used for medical procedures and/or for mapping itself, as described below.

The plurality of voltage measurements v_{(i,j)} between pairs i, j of electrodes, performed as described above, when a plurality of different excitations is applied over time to a plurality of electrodes and measured by a plurality of electrodes, creates a collection of a plurality of data sets V_{(i,j)} of voltage measurements. For example, each voltage measurement v(ij) can be seen as a data set and the collection V(ij) hence represents a plurality of such data sets. The collection V_{(i,j)} of voltage measurements may be obtained when the intra-body electrodes are located at different positions within the body (e.g., as the catheter moves inside an organ). The data sets and/or the collection may additionally include values indicative of currents applied to excite electrodes i and/or position data indicating the position of the electrodes i and j in a reference frame, for example fixed on the catheter or on the body. Alternatively, these values may be accessed separately, for example from a different data structure, or they may be recoverable from known information about currents and position, based on a known association between these values and electrode indices or even sequence of appearance of the data in the data set or collection. Specifically, the same currents may be applied to all electrodes i.

Each voltage measurement v(ij) can be seen as a data set and the collection V(ij) hence represents a plurality of such data sets. In the example above, each data set is defined for a pair of electrodes, one having current applied to it and the other one used to measure a voltage. It will be appreciated that the present disclosure is applicable more widely and equally applies to pairs of sets of electrodes, one set having currents applied to it and one set used for measuring voltages. Where the disclosure refers to single electrodes for current application or voltage measurement, it will be understood that respective sets of electrodes may equally be used.

The collection of voltage measurements may be converted to a collection of spatial conductivity values, that is a spatial distribution σ_{(x,y,z)} of conductivity, assigning a calculated conductivity value to points in a defined 3D volume, as is known to the person skilled in the art based on the laws of electromagnetics, for example as described below. The points in the distribution σ_{(x,y,z)}, with their assigned conductivity values may be included in a large collection (or a cloud) of spatial values, hereinafter denoted R and represent a map of dielectric properties, specifically conductivity, in the region covered by R.

It will appreciated that the body volume that may be mapped may be defined as a body volume confined between/among a set of surface electrodes usable in the imaging process. However, the mapped volume need not be defined in this way but can extend to all points where sufficient information is available from the measurements taken to compute σ_{(x,y,z)}. Indeed, surface electrodes need not even be present, as described above.

In practice, the intra-body electrodes are typically disposed on a catheter or other tool, so they may move with the catheter inside the body, when the catheter is moved, e.g. along a body lumen or inside a heart chamber or other organ(s). Solving the 3D conductivity map (i.e. calculating the spatial distribution of conductivity value for the collection of 3D points in the scanned volume of the body based on voltages measured at the surface of the imaged volume and inside it or around it) may not require knowledge of the position of the electrodes, (other than knowing which are at the surface and which are inside the body), but the solution depends on that location.

It will be appreciated that excitation schemes may vary in terms of the placement and identity of electrodes used. In some embodiments, both surface and intra-body electrodes are used. In some embodiments, the intra-body electrodes are disposed on a moveable catheter or tool, which is moved from one position to the next to acquire respective sets of data. In some embodiments, two or more sets of intrabody electrodes are used, each disposed on a respective catheter. At least one of the catheters is stationary, providing a reference frame fixed to the body as in the case of the surface electrodes, and at least one of the catheters moves during data acquisition. In more general terms, in some embodiments, data is collected using one stationary set of electrodes substantially fixed in relation to the body and one moving set of electrodes, moving from one position to the next. In some embodiments, all electrodes are disposed on a moving catheter and no stationary electrodes are used.

A subset of the electrodes will be used to generate an electric field and another subset of the electrodes will be used to measure at any one time. The generating and measuring electrodes can, in accordance with different arrangements be distributed in any suitable manner between the sets of electrodes. Particular mapping techniques involving the combination of locally obtained frames of measurement are applicable to embodiments where both the emitting and measuring electrodes are disposed on a moving catheter and will be described in more detail below. In some embodiments, surface electrodes can also be taken into account in obtaining local frames based on the position of the surface electrodes in a frame of reference fixed on the catheter. In some arrangements, both measuring and emitting electrodes are surface electrodes and a catheter, with or without electrodes, is used to provide constraints to the map reconstruction based on its known spatial distribution of dielectric properties.

Reference is made now to FIG. 3 which schematically depicts electric field generator/measurer 300. Field generator/measurer 300 of FIG. 3 enables two electrodes to be configured to transmit each at a different frequency, and receive (and measure) at this frequency, and at the frequency transmitted by the other electrode. Signal source 310 provides signal in frequency f1. This signal is fed to electrode, e.g., electrode 210 (of FIG. 2) via terminal point 350 and the signal reaches another electrode, e.g., electrode 212 (of FIG. 2) and received by it. Similarly, signal source 320 provides signal in frequency f2. This signal is fed to electrode 212 via terminal point 360 and the signal reaches electrode 210 and received by it. As a result, junction points 301 and 302 experience a multiplexed signal comprised of frequencies f1 and f2. D is a demultiplexer that is configured to receive, in the current example, multiplexed signal (comprising signals in frequencies f1 and f2) and enable only signal in one of the frequencies to pass through—signal in frequency f1 passes via D 332 and D 344 and signal in frequency f2 passes via D334 and D 342. Accordingly, voltmeter 312 measures the amplitude of the signal in frequency f1, as originated from signal source 310 and received by electrode 210, and voltmeter 314 measures the amplitude of signal in frequency f2 as originated from signal source 320 and received by electrode 210. The demultiplexing of the signals at section 300B of electric field generator/measurer 300 is done in the same manner, where 320 is the signal source of the signal having frequency f2, and 322 and 324 are the voltmeters, measuring signals at frequencies f2 and f1 respectively.

It will be apparent that for exciting more electrodes the sections 300A, 300B of electric field generator/measurer 300 may be repeated. In some embodiments, other signal demultiplexers may be used, as is known in the art.

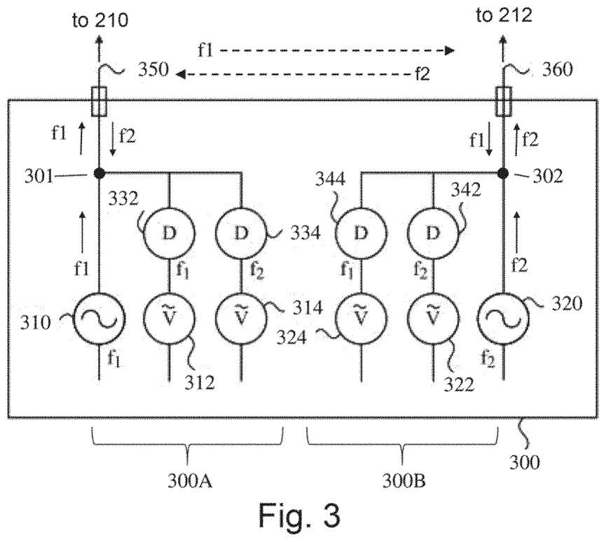

Reference is made to FIG. 4, which is a schematic block diagram of system 400 for dielectric—mapping and/or imaging. Specifically, in some embodiments, the system 400 is configured to implement the methods disclosed in this application. System 400 may comprise main control unit 402 in active communication with surface electrodes unit 410 (where present) and intra-body electrodes unit 420 (where present), via communication channels 410A and 420A1 respectively. Main control unit 402 may comprise controller 404 and signal generator/measurer 406, connectable via electrodes I/O interface unit 408. Control unit 402 may include a controller that may be, for example, a central processing unit processor (CPU), a chip or any suitable computing or computational device, equipped with an operating system, a memory, an executable code, and a storage (not shown in order to not obscure the drawing). Main control unit 402 may be configured to carry out methods described herein, and/or to execute or act as the various modules, units, etc. More than one computing device may be included in the system, and one or more computing devices may act as the various components of the system. For example, by executing the executable code stored in the memory, the controller may be configured to carry out a method of acquiring signals from the electrodes for the construction of a 3D map.

Signal generator/measurer 406 may produce signals in a manner similar to the description of the signals produced and measured by generator/measurer 300 of FIG. 3. Accordingly, signals may be fed to, and/or received from any of the body surface electrodes of surface electrodes unit 410 and intra-body electrodes of intra-body electrodes unit 420. Body surface electrodes of unit 410 may be deployed and operated similarly to electrodes 102A/102B, 104A/104B 106A/106B of FIG. 1. Intra-body electrodes of unit 420 may be arranged and operable similar to electrodes 210, 212, 214 and 216 of FIG. 2A or corresponding electrodes of FIG. 2B.

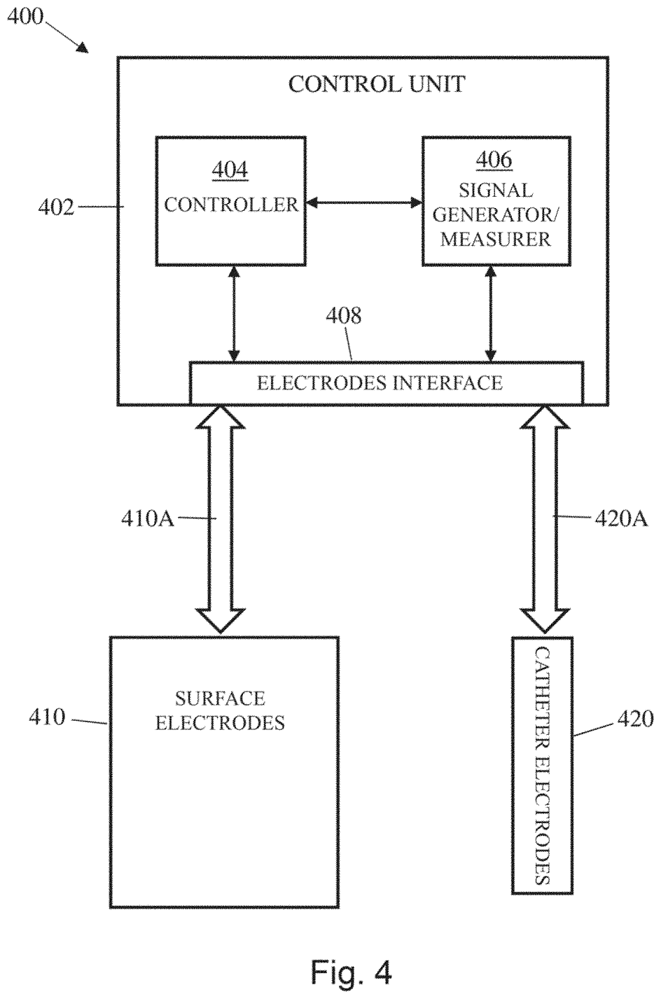

Reference is made to FIG. 5, which is a top-level flow of process 500 for converting a collection of measured voltages on a set of electrodes into a dielectric map and, in some embodiments, a 3D image. A plurality of electrical signals may be injected to the electrodes, surface electrodes and/or intra-body electrodes, according to one or more excitation schemes, as discussed above. A plurality of measured voltage data sets j) (502), measured at the plurality of electrodes, may be combined into a collection of a plurality of data sets V(i, j) (504) as described above, which then may be converted (or reconstructed) into large number of conductivity values, each of which is associated with a 3D point having a respective x, y, z spatial coordinates (508), thus defining a spatial distribution σ_{(x, y, z)} (506) or dielectric map. Optionally, the collection of spatial conductivity values (the map) may then be translated into a 3D image (510) that may be presented on a display or otherwise presented. The translation may be based on assigning a pseudo-color or grayscale value to each conductivity value or by assigning ranges of conductivity values to corresponding tissue types, for example.

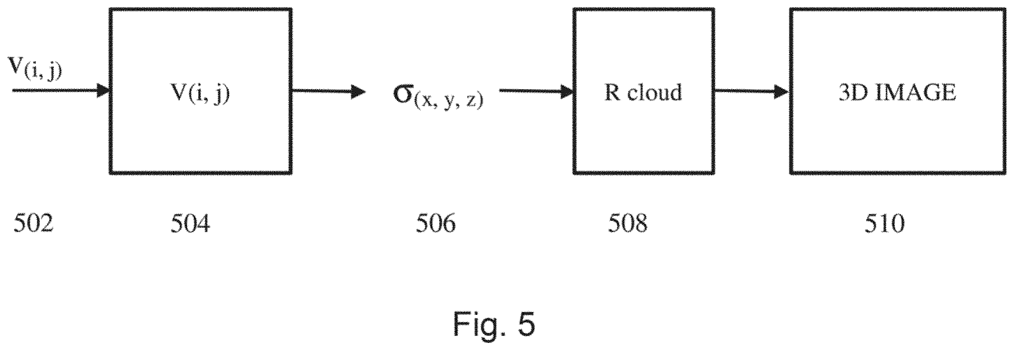

Reference is made to FIG. 6, which is a flow chart depicting method for dielectric mapping, optionally for imaging a body volume or for reconstructing body volume.

The body volume may include or be a body tissue. Currents may be injected at block 602, for example by control unit 402 using signal generator/measurer unit 406, to electrodes deployed on a patient's body, such as electrodes 410 of FIG. 4 (for example, electrodes 102A/B, 104A/B and 106A/B of FIG. 1), and/or to intra-body electrodes, such as electrodes 420 of FIG. 4, for example electrodes 210, 212, 214 and 216 of FIG. 2, according to an injection scheme (block 602). Injection schemes may include a time/frequency transmit scheme. Injection schemes may be controlled and monitored by controller 404. At block 604, voltages are measured on electrodes (e.g., on all electrodes) e.g. by signal generator/measurer 406, and an inverse problem (calculation and production of 3D spatial distribution of conductances of body tissues based on the currents/voltages measured) (block 606) may be solved, e.g. by control unit 402, and a 3D conductance map (3D distribution of conductance measurements, also referred to herein as conductivity map) may be obtained and optionally provided for display (block 608). At block 610, a 3D image of the body tissue may optionally be produced (and optionally presented) based on the 3D conductance map.

It will be appreciated that the method may include a precursor to step 602 of placing the surface electrodes (if used) on a patient and of inserting the intrabody electrodes into the patient. However, in some embodiments, the method excludes any surgical steps and is limited to receiving data sets values indicative of currents applied to the excitation electrodes (for example current values, electrode charge values, electric field values at the electrode in question) and of values indicative of voltage measured at the measurement electrodes (for example voltage values, current values, impedance values, electric field values) and performing the disclosed data processing on the received data sets to generate a dielectric map and, optionally, an image based on the dielectric map.

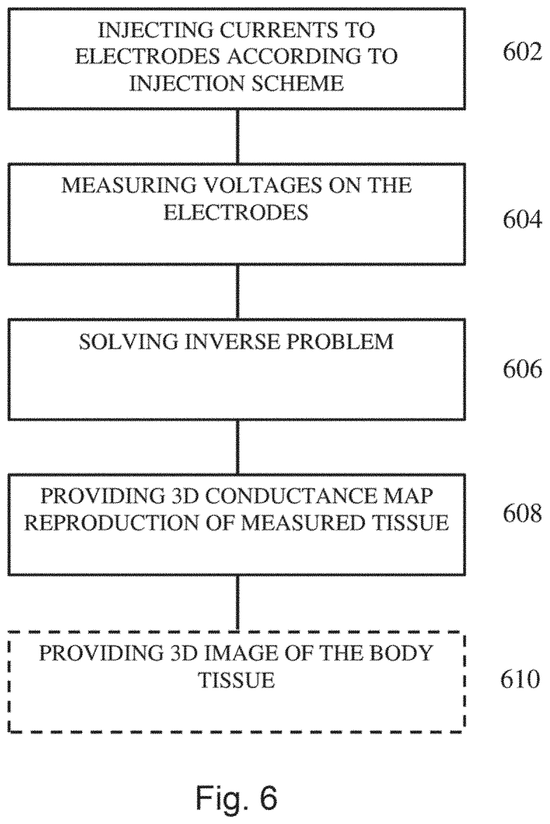

The methods referred to above generically refer to solving the inverse problem, that is, to finding a spatial distribution of conductances (or other dielectric quantities) given spatially located field sources (resulting from injected currents) and spatially located field (voltage) measurements. Many different approaches to solving this problem are known, some of which involve a form of optimization to find a spatial distribution of conductances consistent with the field sources and measurements. For example, with reference to FIG. 7, a model of the spatial distribution of conductances σ_{(x, y, z)} may be initialized to a starting guess and then optimized to be consistent with a set of current values I₍ᵢ₎, where i designates an electrode at a known position in a reference frame and I is a value indicative of the current applied to that electrode, and a set of voltage values v_{(i,j)} indicative of a measured voltage at electrode j of known position in the reference frame in response to current applied to electrode i. The current values I₍ᵢ₎ may be fixed parameters known in advance, for example set to a fixed value of magnitude and frequency of a current waveform, in which case I₍ᵢ₎ is applicable to all data sets v_{(i,j)} or may vary, in which case respective values of I₍ᵢ₎ are included in the data set. The current values can be the known or measured values of currents applied to the electrode, or measurements of currents running through the electrodes. The voltages and currents may be real valued (for example if real-valued conductance is mapped) or may be complex-valued (for example if complex conductance or admittance is mapped).

The method comprises receiving 702 the collection V_{(i,k)} of a plurality of data sets v_{(i,k)} and I₍ᵢ₎ and initializing 704 an initial “guess” of σ_{(x, y, z)}. The initial guess may be random, may be based on knowledge of the anatomical structure, or may be based on a previously calculated σ_{(x, y, z)} calculated under related conditions, as described in more detail below. Modeled values V*_{(i,j)} of measured voltages are calculated 706 using physics knowledge, for example Maxwell's equations or Laplace equations, applied to the current values I₍ᵢ₎(or I if fixed and predefined), the known positions of the electrodes i and j and the present σ_{(x, y, z)}, for example the initial guess on the first iteration. An error signal is computed 708 as a function of the magnitude of the difference between measured and modeled voltage values. The function may be simple, for example the absolute or squared difference, or may include further terms to guide optimization, for example based on soft constraints as discussed in detail below, or for example based on the entropy of σ_{(x, y, z)}, as is well known in the art of function optimization. The error signal is used to adjust 710 σ_{(x, y, z)} using gradient descent on a gradient of the error or other well-known optimization techniques (treating the parameters defining σ_{(x, y, z)} as the optimization parameters to be optimized). Before or after updating σ_{(x, y, z)}, the method involves checking 712 whether a stopping criterion has been met, for example in terms of the error signal falling below a threshold value or changing by less than a threshold amount compared to the previous iteration(s). If the stopping criterion is not met, the method circles back to computing 706 modelled voltages and otherwise stores 714 σ_{(x, y, z)} and either terminates or proceeds to optional processes, such as computing 716 a medical image based on σ_{(x, y, z)}.

Numerous ways of defining σ_{(x, y, z)} are envisaged. In one example, σ_{(x, y, z)} is defined in terms of a linear superposition of base conductance distributions for a target organ to be mapped that have been derived before by other means, for example other optimization techniques or based on other imaging modalities across a group of subjects. In this case, the optimization parameters are the superposition coefficients and optimization is based on numerically calculated gradients or other means, such as Monte Carlo methods. In another example, σ_{(x, y, z)} is defined on a mesh of conductances and Finite Element Analysis (FEA) is used to calculate the forward model (V*). In some embodiments the mesh may be a uniform cartesian mesh defined in terms of x, y and z axes, while in other embodiments a non-uniform tetrahedron mesh is used, adjusted based on the locations of the electrodes (and hence the location of the available information), as is well known in the field of FEA. Where multiple frames of measurement are obtained, the mesh may be determined dynamically and optimized in each instance or, in embodiments that favor efficiency, a mesh may be predefined, for example based on catheter electrode configuration, for all frames. Irrespective of how the mesh/cells of the FEA model are defined, in some embodiments the (tetrahedron) conductance values of the FEA model are the optimization parameters adjusted based on the error signal.

The optimization problem of finding σ_{(x, y, z)} is a difficult one in that in order to achieve desirable levels of resolution, many parameters need to be adjusted based on data from an inevitably limited number of electrodes. While various regularization approaches are known to help with this problem, the inventors have realized that it is possible to use known dielectric characteristics of a catheter or other tool placed in the region to be mapped to constrain the optimization. This approach is applicable irrespective of the identity of the electrodes used for field generation and measurement and may, for example, be applied to embodiments in which only surface electrodes are used for both measurement and field generation. In these cases, the catheter is placed in the region merely to provide constraint data without participating in the measurement. Evidently, in other embodiments in which intrabody electrodes participate in field generation or measurement, the catheter may have a dual function of carrying the intrabody electrodes and providing constraint data. In some embodiments, constraint elements not on the catheter carrying intrabody electrodes may be used, for example dielectric or conductive parts on other tools disposed in the body, conductive or dielectric markers permanently or temporarily secured to the body or organ and so forth.

The known information about the catheter (or other known body) may take various forms, for example: a distribution of the dielectric properties of the catheter, such a distribution combined with a known position of the catheter in an external reference frame (for example defined by the surface electrodes), a length and known dielectric properties of a plastic part of the catheter, a position and/or configuration of electrodes on the catheter, a distance between electrode pairs on the catheter, the position of metal elements such as electrodes on the catheter that are or are not used for field generation or measurement and the like. These and other items of information about the catheter will be most informative when available in the same reference frame as the measurements. For example, this would be the case for measurements made with the surface electrodes, where the position of the catheter is known within the reference frame of the surface electrodes fixed to the body. Position detection of the catheter may be by external means, such as medical imaging, for example computer tomography or magnetic resonance imaging, or as described further below. This would also be the case where measurements are taken in the reference frame of the catheter itself that is where the emitting and measuring electrodes are both disposed on the catheter, and the constraints are defined on the catheter, as well. However, some measurements such as distance measurements between landmarks such as electrodes on the catheter are invariant to the frame of reference and such constraints can be used irrespective of the frame of reference, by detecting the landmarks in the current iteration of σ_{(x, y, z)} and using this to constrain the optimization.

The constraints may be used to influence the optimization discussed above as soft or hard constraints, as is known in the art. A soft constraint is provided by adding an additional term punishing deviations from the constraint to the function defining the error signal computed at step 708, so that the resulting gradients (in the case of gradient descent) are biased towards solutions that are consistent with the constraint. For example, where a distribution of dielectric properties is known in the frame of reference of reconstruction, such as when all electrodes are provided on the catheter and the distribution of the dielectric properties of the catheter are used as constraint, the function defining the error signal may comprise a term penalizing the magnitude of deviation of σ_{(x, y, z)} from the known dielectric distribution in the region of the catheter, averaged over the catheter. In addition or alternatively, for example, the function may comprise a term penalizing a deviation from the know distance between electrodes detected as landmarks in σ_{(x, y, z)}, or between other landmarks. Implemented as hard constraints, the adjustment at step 710, in some embodiment, is altered to include an additional adjustment in addition to the optimization update. The additional adjustment ensures that after step 714 σ_{(x, y, z)} meets the constraint and may, for example, include, in the region where constraints are defined in terms of a dielectric distribution, setting values of σ_{(x, y, z)} to that dielectric distribution, or scaling, rotating or otherwise transforming σ_{(x, y, z)} to be consistent with distance-based constraints, as the case may be.

In addition to the above-described examples for solving the inverse problem, that is finding a spatial distribution of conductances (or other dielectric quantities) given spatially located field sources (resulting from injected currents) and spatially located field (voltage) measurements, some other approaches involve using machine-learning techniques to determine a spatial distribution of conductances. In general, a function may map measurements (measured voltage data and position data) to a spatial distribution of conductances or other dielectric map. The function may be an artificial neural network that takes the measured voltage data and position data as input, and provides the spatial distribution as output. The function may also be a lookup table that finds a spatial distribution based on measured voltage data and position data.

In more detail, instead of the backward-system methods described above, a forward system approach may be used to train a machine learning model, which can then be used to retrieve or determine a conductivity (or other dielectric property) map based on measured voltages, without the need to perform the optimization processes discussed above. The model can be trained by simulating measurements that would be measured for a number of sample images of a structure (each image being imaged under a known imaging condition, for example with a known relation (angle, distance) between the tool and the structure), the simulated measurements being determined using a forward model based on the image and imaging conditions to obtain a training data set. The training data set preferably comprises plural imaging conditions and the training data preferably includes a representation of the imaging conditions. The simulated measurements can be stored in a lookup table that associates these measurements with the corresponding sample image and imaging conditions, where applicable. The lookup table may be used to train an artificial neural network to output the conductivity or other map given actual measurements and, where applicable, imaging conditions. The output may be directly a map, or the output may be a classification score for each of a number of representative maps. In the latter case, the classification score can then be used to retrieve a representative map (for example the highest scoring one) or to form a weighted average of representative maps based on the respective classification scores. Alternatively, the lookup table can directly be used to associate new measurements taken using the electrodes to an image in the lookup table, by identifying an entry in the table that is closest to the new measurements, thereby identifying an image that is similar to an image that should be associated with the new measurements. Alternatively, several entries close to the new measurements may be identified, and the corresponding images may be interpolated to obtain a new image that corresponds to the new measurements.

In some of the described embodiments, measurements are made and fields generated with moving intrabody electrodes. For example, the electrodes may be disposed on a moving catheter or other tool. As the intrabody electrodes move from location to location, respective frames of measurements and corresponding spatial distributions are generated. The electrodes used for the measurements and corresponding field generation may be only on the catheter or include electrodes disposed in a fixed relationship to the body (fixed electrodes), such as described above. For combining information from fixed and moving electrode, the locations of the fixed electrodes may be transformed into a common moving frame of reference common with the intrabody electrodes and moving with the catheter. In either case, a sequence of dielectric maps (or frames) is generated corresponding to locations through which the catheter travels. These maps are, in some embodiments, combined to obtain combined map of the region of interest through which the catheter travels.

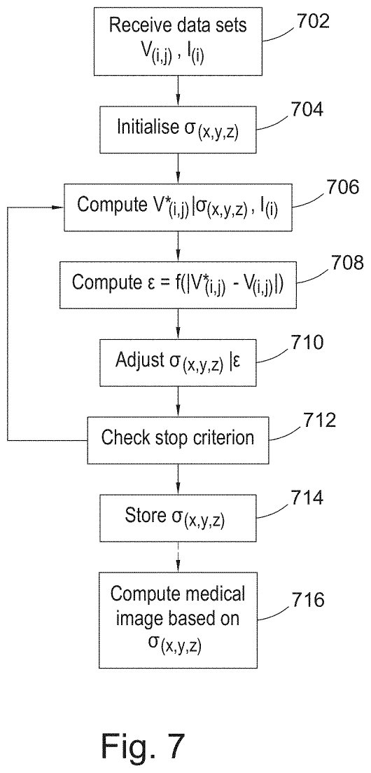

With reference to FIG. 8, two or more maps are computed, displacements between them are determined, and the two or more maps are combined. In the figure, combining two maps is described in detail, but adding to the process further maps is possible, e.g., by looping back from before step 808 to step 802 (generating a fresh pair of maps to be combined) or 804 (combining a previously generated map with a newly generated map). In some embodiments a first map is computed 802 for a first catheter location and a second map is computed 804 for a second location. Between steps 802 and 804 the catheter may be moved from the first to the second location to acquire the data for the computation of the second map, or the data acquisition may have happened at a previous time at the first and second location (or even at all location used) of the catheter. In the latter case, a processor such as the control unit 402 receives the previously acquired data sets for each corresponding catheter position from a database.

A displacement between the first and second locations of the catheter is computed 806, as described in more detail below, and the first and second maps are combined 808 based on the computed displacement. The displacement may be computed as a linear translation between the two maps, for example a displacement vector (or equivalently a diagonal displacement matrix corresponding to the displacement vector), or by a translation and rotation, for example encoded in a displacement matrix with appropriate off-diagonal entries. Combining the first and second maps may, for example, involve averaging the two maps together in the region of overlap (optionally rotated as appropriate) between the two maps, as determined by the computed displacement. Other ways of combining the maps are of course equally possible, for example, picking the values of one map in any region of overlap. It will be appreciated that in these examples the order of the steps is not important, as long as the two maps and the displacement are available to combine the two maps at step 808.

Subsequent to step 808, further maps, as well as further corresponding displacements may be computed and combined. In some embodiments, a larger number of individual maps are calculated, as well as corresponding mutual displacements and these are then used to produce combined maps. The process is thus not limited to merely combining two adjacent maps (maps captured at adjacent locations of the catheter) but a number of overlapping maps can be combined to compute individual combined maps. Irrespective of how the combined maps are derived, the combined map may be computed for the respective regions of overlap only or may also include non-overlapping regions. The individual combined maps may then be stitched together to provide a map that covers more than one catheter position and covers some or all of the track of the catheter through the organ, as illustrated in FIG. 8A in one particular example, in which the shaded region indicates a region of increased resolution along the track of the catheter, where the combined map benefitted from the overlapping data from two or more individual maps. Numerous techniques for combining maps are available to the person skilled in the art, for example from the field of image processing, adapting techniques for the combining and/stitching together of images, for example super resolution techniques, for use with the 3D spatial distributions or maps of the present disclosure.

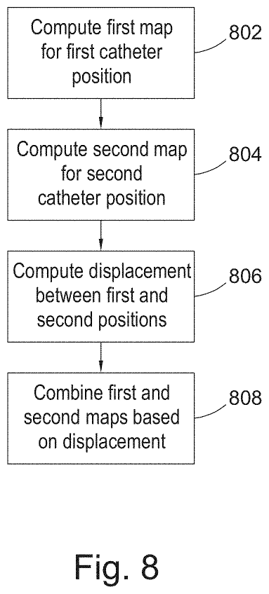

In some embodiments, now described with reference to FIG. 9, a first map is computed for a first location and used in the computation of the second map, for example using the first map to initialize the second map at step 704 of the map computation process described above with reference to FIG. 7. It will be appreciated that this process can be combined with that in FIG. 8 described above in that the resulting maps can then be combined or averaged as described above. In any event, the resulting maps can be stitched together to form a composite map, as illustrated in FIG. 8A.

Specifically, a first map is computed 902 for a first catheter location and a displacement is calculated 902 between the first catheter location and a second catheter location to which the catheter has moved. As described above, the catheter may be moved between steps 902 and 904 or the first and second locations may correspond to respective data sets in a database of pre-acquired data sets at different catheter locations. The second map is then computed 906 based on the first map and the displacement. For example, a portion of an initial guess of the second map may be set to the region overlapping between the first and second maps, with the region of overlap determined based on the displacement (with or without a rotation applied as discussed above). Outside the region of overlap, the second map may be initialized with random values or in any other suitable way.

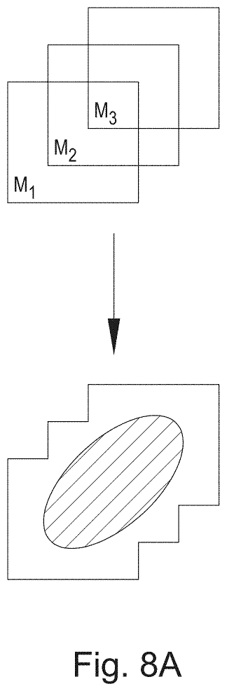

Various techniques for computing a displacement (with or without rotation) between the first and second maps in the above processes are now described. It will be understood that these techniques may be useful in their own right to compute displacements between catheter positions for reasons other than to determine the overlap between maps, in the context of combining maps or otherwise. With reference to FIG. 10, a process for computing a displacement matrix (or vector) D comprises computing 1002 the multidimensional cross-correlation between the respective maps (spatial distributions) M1, M2 corresponding to the first and second locations. In the case of a pure displacement or translation, the cross-correlation function would be three-dimensional (one for each direction in Cartesian space, for example), whereas a displacement matrix allowing for some or full rotation to be captured would have up to 9 dimensions to capture the corresponding affine transformation. Subsequently, an indication of displacement between the maps, being the displacement at which the cross-correlation exceeds a comparison value (for example the displacement for which the cross-correlation has a maximum value) can be found. Specifically, a displacement vector or matrix Dmax at which the cross-correlation is at a maximum is found 1004 and Dmax is applied to M1 to displace M1 into alignment with M2 and the result is combined 1006 with M2. Combining M1 and M2 may comprise averaging M1 and M2, or M1 may be used as a starting point for a re-calculation of M2. A bootstrap procedure may be used by which M1 is used as a starting point for re-calculating M2, then M2 is used as a starting point for M1 and so forth until M1 and M2 converge to a respective value. Using one map as a starting point for calculating another map has been described above. Whilst in this example the displacement vector or matrix Dmax is the displacement at which the cross-correlation is at a maximum value, the displacement vector/matrix may be the displacement at which the cross-correlation exceeds any other threshold, otherwise referred to as a comparison value.

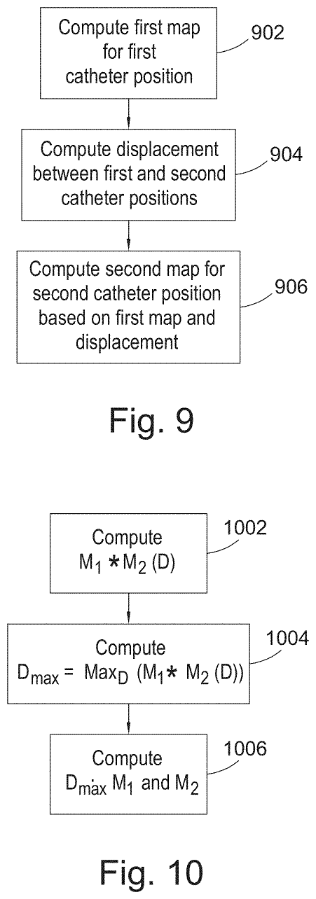

The above description of combining a displaced version of a first map with a second map in the region of overlap between the first and second maps is applicable in a straight forward manner if the first and second maps are defined on a uniform, common, mesh so that the displacement calculated for the first map is meaningful in terms of the mesh of the second map. However, as described above, where the maps are calculated using FEA, a uniform or regular mesh will often be sub-optimal, as in many cases it does not reflect the distribution of information available to constrain the FEA. As a consequence, a non-uniform mesh is often used to define the map for the purpose of the FEA. In such cases, or other cases in which the meshes of the two maps differ from each other, the displacement between the first and second positions of the catheter cannot be directly applied to the first map. With reference to FIG. 11, a process to deal with this, which may for example be incorporated with steps 808, 906 and 1006, comprises projecting 1102 the first map onto a regular mesh, for example a Cartesian mesh, applying 1104 the displacement to the projected map and projecting 1106 the result to the mesh in which the second map is defined. Alternatively, both maps may be projected onto a common, regular mesh for the purpose of combination.

Computing correlations as described above, requires the maps to have sufficient structure and/or contrast in their values so that the correlation peak is sufficiently sharp to enable a desired level of confidence in the computed displacement. An alternative method uses three or more pairs of surface electrodes (or other static electrodes such as may be provided on a stationary catheter) to generate electric fields, the gradients of which are used to calculate local displacements as discussed below. The electric fields generated by the pairs of electrodes are mutually non-parallel, for example mutually orthogonal, to set up a corresponding coordinate system. Equally, the fields (or currents generating them) are separate either in time or in frequency, so that separate field gradient can be calculated for each field and corresponding gradient direction.

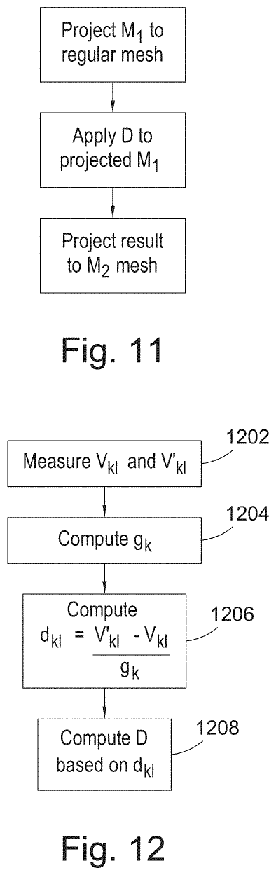

With reference to FIG. 12, in some such embodiments, a number of voltage measurements V_{k,l} are taken 1202 using a number of respective spaced apart electrodes on the catheter at respective locations. For these measurements pairs of voltages V_{k,l} and V′_{k,l} measured at a corresponding pair of electrodes can be defined. It will be appreciated that in methods that are not carried out online, this step may be replaced with a step of accessing previously measured values in a database. For example, the electrodes may be spaced along a direction of travel of the catheter, as illustrated in FIG. 2A, or define a subset of electrodes that are spaced along a direction of travel of the catheter, for example in an arrangement as in FIG. 2B. The electrodes (and hence their position along the catheter) are indexed by land the gradient electric field (and hence the corresponding direction) is indexed by k.

A local voltage gradient gₖ is calculated 1204 for each gradient field based on the configuration of (distance between) the 1 electrodes. Based on the difference between corresponding voltages V_{k,l} and V′_{k,l} recorded at respective catheter positions and the calculated gradients gₖ, corresponding local displacements are calculated 1206 in a linear approximation as

$${d}_{k , l} = \frac{{V}_{k , l}^{′} - {V}_{k , l}}{{g}_{k}} .$$

A displacement D is then calculated 1208. Depending on the calculation and the placement of the electrodes used, D may be calculated as a diagonal matrix or displacement vector by averaging d_{k,l} over l and using the resulting values (or a linear combination thereof) as entries in the diagonal matrix or vector. Alternatively, a full displacement matrix accounting for changes in orientation may be constructed using knowledge of the configuration of the/indexed electrodes and the respective d_{k,l} displacements between them.

Other alternative techniques for combining local maps generated based on voltage measurements at various positions of a moving catheter involve locating each respective position of the catheter in a frame of reference fixed with respect to the body and then either to combine the respective maps in that frame of reference or use that frame of reference to calculate displacements between maps, possibly with suitable mesh transformations, as described above. Such alternative techniques may involve computing electrical impedance tomography images or other dielectric maps using time varying electric fields generated by surface or other static electrodes, for example disposed statically inside the body, and locating the catheter in these images, for example by detecting dielectrically salient features or landmarks on the catheter, such as the electrodes disposed on the catheter. Another alternative example is to set up at least three non-parallel electric fields separated in time or in frequency and using a pre-computed mapping from local voltages measured on the catheter to catheter positions to find the required catheter positions.

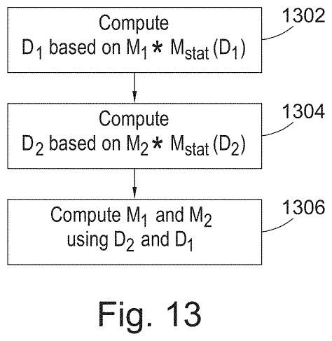

Yet a further example that employs surface electrodes, or other electrodes disposed in a fixed relationship with the body, for example disposed on a static catheter disposed in the vicinity of the moving catheter, computes the required displacements between maps using cross-correlations with a static conductance map calculated using fields generated by static electrodes. For example, the static catheter may be disposed in the coronary sinus for imaging the left or right atrium. With reference to FIG. 13, a first displacement D1 between the first map M1 and the static map Mstat is computed 1302 using a cross-correlation as described above for cross-correlation between local maps. Likewise, an analogous displacement D2 is calculated 1304 between the second map M2 and the static map Mstat. D1 and D2 are then used to combine 1306 M1 and M2, for example by computing a displacement D between M1 and M2 in the M2 frame of reference or even in the frame of reference of Mstat, fixed relative to the body.

It is expected that during the life of a patent maturing from this application many relevant intra-body probes will be developed; the scope of the term intra-body probe is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

1-2. (canceled)

3. A method of combining first and second spatial distributions of dielectric properties in a region of an organ of a human or animal body, wherein each of the first and second distributions was obtained based on measurements from electrodes on a tool positioned in respective first and second positions in the region, the method comprising:
accessing the first and second distributions,
computing an indication of a displacement between the first and second positions of the tool based on measured gradients of electric fields in the region measured using the electrodes; and
combining the first and second spatial distributions using the indication of the displacement, wherein the measurements from the electrodes comprises measurement of voltages generated by an electric field generated by alternating electrical currents applied to at least one electrode on the tool.

4. The method of claim 3, wherein combining the first and second spatial distributions comprises using correspondence between locations in the first spatial distribution and locations in the second spatial distribution.

5. A method according to claim 3, wherein combining the first and second spatial distributions comprises combining values of the dielectric properties at respective locations in the second spatial distribution with values of the dielectric properties at corresponding respective locations in the first spatial distribution.

6. A method according to claim 4, comprising determining the correspondence between locations in the first spatial distribution and locations in the second spatial distribution using the indication of the displacement.

7. A method according to claim 3, wherein computing an indication of displacement comprises computing a cross-correlation between the first and second spatial distributions and determining the indication of displacement between the first and second spatial distributions as the displacement at which the cross-correlation exceeds a comparison value, preferably the displacement for which the cross-correlation has a maximum value.

8. A method according to claim 3, the method comprising using the first spatial distribution as a starting distribution in an iterative process reducing an error between predicted and actual measurements to compute the second spatial distribution.

9. (canceled)

10. A method according to claim 3, comprising computing the indication of the displacement using data collected from electrodes placed in a fixed relationship to the body.

11. A method according to claim 10, wherein the data collected from electrodes placed in a fixed relationship to the body comprise voltages recorded at the electrodes placed in a fixed relationship to the body in response to currents applied to electrodes placed in a fixed relationship to the body.

12. A method according to claim 10, where in the electrodes placed in a fixed relationship to the body are disposed on the body and/or on a tool that has been placed in a stationary position inside the body, preferably inside the organ.

13. A method of combining first and second spatial distributions of dielectric properties in a region of an organ of a human or animal body, wherein each of the first and second distributions was obtained based on measurements from electrodes on a tool positioned in respective first and second positions in the region, the method comprising:
accessing the first and second distributions,
computing an indication of a displacement between the first and second positions of the tool;
combining the first and second spatial distributions using the indication of the displacement, wherein the measurements from the electrodes comprises measurement of voltages generated by an electric field generated by alternating electrical currents applied to at least one electrode on the tool;
determining the respective positions of the tool at the first and second positions in a reference frame fixed relative to the body;
and determining the indication of the displacement using the determined positions by:
computing respective global spatial distributions of one or more dielectric properties in a portion of the body including the region when the tool is positioned in the first and second position, wherein the global spatial distributions are defined in a frame of reference fixed to the portion of the body; and
determining the respective positions using the global spatial distributions.

14. (canceled)

15. A method according to claim 13, wherein determining the respective positions comprises analyzing each of the global spatial distributions to detect one or more electrodes on the tool in each of the global spatial distribution and determining the respective positions using the positions of the one or more electrodes in the respective global spatial distribution.

16. A method according to claim 13, wherein determining the respective positions comprises:
computing cross-correlations between each of the first and second spatial distributions and the respective global spatial distribution;
determining the position of the tool at the respective location using the displacement between the respective one of the first and second spatial distributions and the global spatial distributions at which the cross-correlation exceeds a comparison value, preferably the displacement for which the cross-correlation has a maximum value.

17. A method according to claim 13 wherein determining the respective positions comprises:
accessing voltage values measured at the electrodes on the tool at the respective positions;
accessing a voltage to position mapping with the respective voltage values to determine the respective positions.

18. A method according to claim 3, wherein the first and second spatial distributions are defined on a respective non-uniform mesh and combining the first and second spatial distributions comprises transforming each of the first and second spatial distribution to be defined on a common mesh having corresponding points in the combined region of the first and second spatial distributions.

19. A method according to claim 18, wherein the common mesh is uniform, in the combined region.

20-41. (canceled)

42. A method according to claim 3, wherein computing one or more spatial distributions comprises:
accessing constraint data characteristic of a spatial distribution of one or more dielectric properties of the tool disposed in the electric fields; and
using the constraint data to compute the one or more spatial distributions.

43. A method of generating a medical image, the method comprising generating a dielectric map using a method according to claim 3, and assigning a tissues type, color or greyscale value to locations in the dielectric map based on the value of the dielectric properties at each of the locations.

44-47. (canceled)

48. A system for generating a dielectric map, the system comprising:
a processor configured to implement a method according to claim 43; and
a memory for storing the dielectric map.

49. (canceled)

50. A system according to claim 48, the system comprising an interface for connecting the system to the electrodes.

51. A system according to claim 50, wherein the processor is configured to cause simultaneous application of currents to some of the electrodes with different frequencies for different non-overlapping subsets of the electrodes.

52. The system of claim 48, further comprising the electrodes.

53. A method according to claim 3, wherein accessing a first plurality of data sets comprises:
(a1) placing a tool in the region, defining a plurality of pairs of sets of electrodes, generating an electric field in the region using a first set of each pair and measuring a voltage at a respective second set of each pair to generate a plurality of data sets; and
(a2) accessing the plurality of data sets, each data set comprising current data indicative of currents applied to the first set of electrodes of a respective pair of sets and measured voltage data indicative of voltages measured at the second set of electrodes of the respective pair of sets.

54. A method according to claim 3, wherein accessing a first plurality of data sets comprises:
(a1) defining a plurality of pairs of sets of electrodes,
generating an electric field in the region using a first set of each pair; and
measuring a voltage at a respective second set of each pair to generate a plurality of data sets; and
(a2) accessing the plurality of data sets, each data set comprising current data indicative of currents applied to the first set of electrodes of a respective pair of sets and voltage data indicative of voltages measured at the second set of electrodes of the respective pair of sets.

55. A method according to claim 3, wherein accessing a plurality of data sets comprises:
(a1) generating an electric field in the region using a first set of each pair of a plurality of pairs of sets of electrodes; and
measuring a voltage at a respective second set of each pair to generate a plurality of data sets; and
(a2) accessing a plurality of data sets, each data set comprising current data indicative of currents applied to the first set of electrodes of a respective pair of sets and voltage data indicative of voltages measured at the second set of electrodes of the respective pair of sets.

56. A method of generating a dielectric map of one or more dielectric properties in a region of an organ of a human or animal body, the method comprising:
(a) accessing a plurality of data sets, each data set comprising voltage data indicative of voltages measured at a respective second set of electrodes in response to electric fields generated by currents applied to a respective first set of electrodes to generate electric fields in the region;
(b) accessing constraint data characteristic of a spatial distribution of one or more dielectric properties of a tool disposed in the electric fields; and
(c) computing the dielectric map as a spatial distribution of one or more dielectric properties in the region using the plurality of data sets and the constraint data.

57. The method of claim 56, wherein at least one of the one or more dielectric properties is selected from the list consisting of: conductivity, complex conductivity, permittivity, and complex permittivity.

58. A method according to claim 53, the method comprising placing the tool inside the body in or in the vicinity of the region.

59. A method of generating a dielectric map of one or more dielectric properties in a region of an organ of a human or animal body, the method comprising:
(a) inserting a tool into the body in or in the vicinity of the region, wherein a plurality of electrodes is disposed on the tool;
(b) defining a plurality of pairs of sets of electrodes of the plurality of electrodes;
(c) generating an electric field in the region using a first set of each pair;
(d) measuring a voltage at a respective second set of each pair to generate a plurality of data sets;
(e) accessing the plurality of data sets, each data set of the plurality comprising measured voltage data indicative of voltages measured at a second set of electrodes of the respective pair in response to the electric field;
(f) accessing position data indicative of positions of the electrodes in the respective first and second data sets relative to the tool; and
(g) computing the dielectric map by using the first plurality of data sets and the position data.

60-71. (canceled)

72. The method of claim 18, wherein the common mesh is Cartesian.
